(19) United States
(12) Patent Application Publication
(10) Pub. No.: US 2006/0193787 A1
(43) Pub. Date: Aug. 31, 2006

(54) Nanoparticle-based drug delivery system

(76) Inventor: Si-Shen Feng, Singapore (SG)

Correspondence Address: DICKSTEIN SHAPIRO MORIN & OSHINSKY LLP, 2101 L Street, NW, Washington, DC, 20037, US

(21) Appl. No.: 11/342,663

(22) Filed: Jan. 31, 2006

Related U.S. Application Data
(60) Provisional application No. 60/648,745, filed on Jan. 31, 2005.

(51) Int. Cl. A61K 9/14; A61L 9/04

(52) U.S. Cl. 424/46; 424/489; 977/906

(57) ABSTRACT

A drug delivery system based on at least one type of nanoparticle. In particular, a nanoparticle-based drug delivery system including at least one first drug, at least one second drug and at least one type of nanoparticle.

### CROSS-REFERENCE TO RELATED APPLICATION

This application claims the benefit of U.S. Provisional Application No. 60/648,745, filed on Jan. 31, 2005, the entirety of the contents of which are hereby incorporated by reference herein.

### FIELD OF THE INVENTION

This invention relates to at least one nanoparticle-based drug delivery system. In particular, the invention relates to a nanoparticle-based drug delivery system comprising at least one first drug, at least one second drug and at least one type of nanoparticle.

### BACKGROUND OF THE INVENTION

Cancer is the leading cause of human mortality. More than 10 million people are diagnosed with cancer every year. It is estimated that there will be 15 million new cases every year by 2020. Although great effort has been made, no substantial progress can be observed in the past fifty years in fighting against cancer. The death rate by cancer in the United States was 1.939% in 1950 and it was still as high as 1.940% in 2001.

Chemotherapy is an effective treatment for cancer and other serious diseases such as cardiovascular restenosis and AIDS. Chemotherapy is a complicated procedure in which many factors are involved in determining its success or failure. It carries a high risk due to drug toxicity, and the more effective drugs tend to be more toxic. Problems still exist even for successful chemotherapy. The patients have to tolerate severe side effects and sacrifice their quality of life. It is believed that the inefficiency and the side effects of chemotherapy are mainly caused by the formulation, pharmacokinetics and toxicity of the chemotherapeutic agents, and the drug resistance of the cancer cells.

Oral delivery of anticancer agents is a challenge in chemotherapy. Except for convenience to the patients, oral delivery of anticancer drugs may greatly improve their efficacy and reduce their side effects. Oral delivery provides a sustained exposure of a safe but effective level of the drug concentration to the cancer cells, which may produce better efficacy and fewer side effects than the current clinical administration, i.e. injection and infusion. Unfortunately, most anticancer drugs are not bioavailable. The bioavailabilty of some chemotherapy drugs such as paclitaxel can be less than 1%. The reason is that the drug would be eliminated by the first metabolic process with cytochrome P450 and the efflux pump P-glycoproteins (P-gp) (Malingre, 2001). Medical solutions for oral chemotherapy, which are currently being developed in some pharmaceutical companies, usually propose to apply P450/P-gp suppressors such as cyclosporine A to make oral chemotherapy feasible. However, the P450/P-gp suppressors would compromise the immune system of the patients and thus cause medical complication to the patients. Also, most P450/P-gp suppressors may have side effects as well as difficulties in formulation of their own.

Further some chemotherapy drugs are hydrophobic and adjuvants used to administer such drugs can have numerous undesirable side effects. To overcome these undesirable side-effects, nanoparticle formulations for oral chemotherapy have been proposed (Feng, 2004).

In nanoparticle formulations of anticancer drugs, biodegradable polymers are commonly used as a matrix to carry the drugs. Research has been focused on the biocompatibility and biodegradation of the polymers. For FDA approved polymers, safety can be guaranteed in general. However, none of them can be perfectly biocompatible. To some degree, the polymers have side effects. Natural polymers such as phospholipids have thus been investigated for nanoparticle formulation of anticancer drugs. However, their degradation and process performance are not as manageable as those of the synthetic polymers (Bummer, 2004).

Accordingly, there is a need in the art for the development of new and/or improved mechanical treatments and/or drug delivery systems for antineoplastic and/or antiproliferative treatments which overcome the limitations and/or problems of the prior art.

### SUMMARY OF THE INVENTION

Accordingly, in one aspect, the present invention provides a drug delivery system. The drug delivery system comprising at least one type of nanoparticle, at least one first drug, and at least one second drug, wherein the nanoparticle and the first drug are contacted and the second drug is applied on the contacted nanoparticle and at least one first drug, and wherein the at least one first drug possesses at least one of the following properties:

(a) emulsifier;

(b) mucoadhesion; or

(c) controls and/or reduces at least one side effect of the at least one second drug.

The contacted nanoparticle and the at least one first drug may form a matrix or part thereof. The at least one first drug may substantially cover the surface of the nanoparticle or the at least one second drug may substantially cover the surface of the nanoparticle.

The at least one type of nanoparticle may be biodegradable and/or bioresorbable. For example, the at least one first drug is MMT, the at least one second may be paclitaxel and the at least one type of nanoparticle may be PLGA. The drug delivery system may be used for delivering at least one drug across a mucosal membrane. For example, the mucosal membrane may be the lining of the gut or respiratory tract.

In another aspect, the present invention provides a method of delivering at least one drug across a mucosal membrane in a subject. The method comprising: providing a drug delivery system comprising at least one type of nanoparticle, at least one first drug, and at least one second drug, wherein the nanoparticle and the first drug are contacted and the second drug is applied on the contacted nanoparticle and at least one first drug, and wherein the at least one first drug possesses at least one of the following properties:

(a) emulsifier;

(b) mucoadhesion or

(c) controls and/or reduces at least one side effect of the at least one second drug;

and

applying the delivery system to the musosal membrane.

The contacted nanoparticle and the at least one first drug may form a matrix or part thereof. The at least one first drug may substantially cover the surface of the nanoparticle or the at least one second drug may substantially cover the surface of the nanoparticle.

The at least one type of nanoparticle may be biodegradable and/or bioresorbable. For example, the at least one first drug is MMT, the at least one second may be paclitaxel and the at least one type of nanoparticle may be PLGA. The drug delivery system may be used for delivering at least one drug across a mucosal membrane. For example, the mucosal membrane may be the lining of the gut or respiratory tract.

Accordingly, the present invention may be provided in a final form of a liquid suspension or mixture, an aerosol spray, a capsule, a tablet, a liquid carrier or a suppository. The present invention may be a kit comprising the drug delivery system of the present invention and information pertaining to its use.

According to a particular embodiment, there is provided A drug delivery system comprising at least one type of nanoparticle, at least one first drug, and at least one second drug, and wherein the at least one second drug is substantially applied on the surface of the nanoparticle and the at least one first drug is substantially applied on the second drug, and wherein the at least one first drug possesses at least one of the following properties:
(a) emulsifier;
(b) mucoadhesion, or
(c) controls and/or reduces at least one side effect of the at least one second drug.
   The at least one first drug may be MMT. The at least one second drug may be a paclitaxel. The at least one type of nanoparticle may be biodegradable and/or bioresorbable. The at least one type of nanoparticle may be PLGA. According to an aspect of this embodiment, the drug delivery system is for delivering at least one drug across a mucosal membrane. Accordingly, there is also provided a method of delivering at least one drug across a mucosal membrane in a subject, the method comprising:
providing a drug delivery system comprising at least one type of nanoparticle, at least one first drug, and at least one second drug, and wherein the at least one second drug is substantially applied on the surface of the nanoparticle and the at least one first drug is substantially applied on the second drug, and wherein the at least one first drug possesses at least one of the following properties:
(a) emulsifier;
(b) mucoadhesion or
(c) controls and/or reduces at least one side effect of the at least one second drug;
and
applying the delivery system to the musosal membrane.
   Abbreviations Used

PLGA: poly(lactic-co-glycolic acid).

PVA: polyvinyl alcohol.

DSC: differential scanning calorimetry.

SEM: scanning electron microscopy.

AFM: atomic force microcopy.

FDA: The US Food and Drug Administration.

PBS: Phosphate buffered saline.

DCM: dichloromethane.

### BRIEF DESCRIPTION OF THE DRAWINGS

FIG. 1. SEM images of the paclitaxel-loaded MMT/PLGA nanoparticles with (a) no MMT, (b) 59% MMT and (c) 100% MMT;

FIG. 2. AFM images of the paclitaxel-loaded MMT/PLGA nanoparticles with (a) no MMT, (b) 59% MMT and (c) 100% MMT;

FIG. 3. DSC thermograms of the pure paclitaxel and the paclitaxel-loaded MMT/PLGA nanoparticles;

FIG. 4. In vitro drug release of the paclitaxel-loaded MMT/PLGA nanoparticles;

FIG. 5. Confocal images of the Caco-2 cells after incubation with 0.25 mg/ml fluorescent nanoparticle suspension for 2 hrs: (a) Control, (b) 50% MMT/NP and (c) 100% MMT/NP.

### DEFINITIONS

Absorbent—The property of a chemical or compound of being able to bind and hold other chemicals.

Biodegradable—The quality of being able to break down in the body of a subject.

Bioresorbable—The quality of being resorbed in or by the body. The terms biosorbable, bioresorbable and bioabsorbable are used interchangeably in the present application.

Carrier, Matrix—the base or vehicle, used in the preparation of pharmaceutical compositions, which is usually the main ingredient of a mixture. The carrier or matrix under the present invention may or may not be inert and may have active pharmaceutical properties. To facilitate application of the pharmaceutical compositions, the carrier or matrix may be further mixed with another pharmaceutical vehicle, depending on the mode of application.

Coating—at least one layer of a chemical or pharmaceutical composition applied to at least one surface of a solid object.

Drug—An active principle, a compound, a medicament and/or a pharmaceutical composition suitable to be administered to a subject to obtain a desirable medical outcome.

Emulsifier—A substance used to produce an emulsion. An emulsion is a preparation of one liquid distributed in small globules throughout the body of a second liquid.

Medical clay—Clays are the common name for a number of fine-grained, earthy materials that become plastic when wet. Chemically, clays are hydrous aluminum silicates. Montmorillonite is a class of clays composed of hydrous magnesium-calcium-aluminum silicate. Medical clays are clays used in medicine that have therapeutical properties.

Mucoadhesive, mucoadhesion—The attraction of a chemical to the mucosal membrane.

Mucosal membrane—The membrane lining internal orifices and containing mucus glands. Mucosal membranes may be found in the gut, respiratory tract and the inside of the eyelids.

Nanoparticle—A particle whose size is in the nanometer range. Under the present invention, a nanoparticle typically ranges from 50 nm to 1,000 nm. In particular, a nanoparticle under the present invention ranges from 100 nm to 800 nm, and more particularly, from 200 nm to 700 nm and more particularly from 200 nm to 400 nm.

### DETAILED DESCRIPTION

Bibliographic references mentioned in the present specification are for convenience listed in the form of a list of references and added at the end of the examples. The whole content of such bibliographic references is herein incorporated by reference.

The present invention relates in one aspect, to a drug delivery system. The drug delivery system comprising at least one type of nanoparticle, at least one first drug, and at least one second drug, wherein the nanoparticle and the first drug are contacted and the second drug is applied on the contacted nanoparticle and at least one first drug, and wherein the at least one first drug possesses at least one of the following properties: emulsifier, mucoadhesion or controls and/or reduces at least one side effect of the at least one second drug.

Under this aspect, the contacted nanoparticle and the at least one first drug may form a matrix or part thereof. The at least one first drug may substantially cover the surface of the nanoparticle or the at least one second drug may substantially cover the surface of the nanoparticle. The at least one type of nanoparticle may be biodegradable and/or biosorbable. For example, the at least one first drug is MMT, the at least one second may be paclitaxel and the at least one type of nanoparticle may be PLGA. The drug delivery system may be used for delivering at least one drug across a mucosal membrane. For example, the mucosal membrane may be the lining of the gut or respiratory tract.

In another aspect, the present invention relates to a method of delivering at least one drug across a mucosal membrane in a subject. The method comprises providing a drug delivery system comprising at least one type of nanoparticle, at least one first drug, and at least one second drug, wherein the nanoparticle and the first drug are contacted and the second drug is applied on the contacted nanoparticle and at least one first drug, and wherein the at least one first drug possesses at least one of the following properties: emulsifier, mucoadhesion or controls and/or reduces at least one side effect of the at least one second drug; and applying the delivery system to the musosal membrane.

Under this aspect, the contacted nanoparticle and the at least one first drug may form a matrix or part thereof. The at least one first drug may substantially cover the surface of the nanoparticle or the at least one second drug may substantially cover the surface of the nanoparticle. The at least one type of nanoparticle may be biodegradable and/or biosorbable. For example, the at least one first drug is MMT, the at least one second may be paclitaxel and the at least one type of nanoparticle may be PLGA. The drug delivery system may be used for delivering at least one drug across a mucosal membrane. For example, the mucosal membrane may be the lining of the gut or respiratory tract.

Accordingly, the present invention may be provided in the form of a liquid suspension or mixture, an aerosol spray, a capsule, a tablet, a liquid carrier or a suppository.

The present invention relates to a first drug which may function as a carrier or matrix for a second drug to facilitate uptake of the drug into cells. The first drug may also have therapeutic effects for preventing, ameliorating or treating another symptom or for preventing, ameliorating or treating any side effects of the encapsulated drug. The first drug may also function as an emulsifier or co-emulsifier for the second drug. Thus the first drug serves any one or more of the three functions above under the present invention.

Under the present invention, the at least one second drug may or may not be encapsulated with the at least one first drug.

It is a new direction that anticancer drugs can be formulated in nanoparticles of another drug, which functions as matrix material or a component of the matrix material of the nanoparticles and may have therapeutic effects for the side effects of the encapsulated drug. It is the purpose of this invention to develop such a novel drug delivery system, in which all elements have their medical function, the encapsulated drug to cure the cancer and the matrix material to treat the side effects caused by the drug, while controlled delivery of both of them can be realized. This invention thus represents a new, non-obvious direction of drug formulation by nanoparticles.

One suitable class of chemicals for use as the first drug used as the matrix or carrier under the present invention is the class of medical clays. A suitable candidate in this class is montmorillonite (MMT)/poly(D,L-lactide-co-glycolide) (PLGA) nanoparticles. The idea is to make use of the mucoadhesive property and therapeutic effects of medical clays such as MMT to make a novel kind of nanoparticle to delivery drugs such as anticancer drugs across a mucosal membrance such as the gastrointestinal (GI) barrier to realize administration of the second drug.

Montmorillonite (MMT) is a member of the general mineral group of the medical clays. Its chemistry is (Na,Ca)(Al,Mg)₆(Si₄O₁₀)₃(OH)₆-nH₂O, hydrated sodium calcium aluminum magnesium silicate hydroxide. MMT typically forms microscopic platy micaceous crystals. The water content is variable and when water is absorbed by the crystals, they tend to swell to several times of their original volume. This makes MMT a useful mineral for biomedical purposes. MMT is a potent detoxifier. MMT could adsorb dietary toxins, bacterial toxins associated with gastrointestinal disturbance, hydrogen ions in acidosis, or metabolic toxins such as steroidal metabolites associated with pregnancy.

All these conditions result in a host of common symptoms, including nausea, vomiting, and diarrhea, most of which are typical symptoms of the side effects caused by chemotherapy. Calcium MMT has also been used extensively in the treatment of pain, open wounds, colitis, diarrhea, hemorrhoids, stomach ulcers, intestinal problems, acne, anemia, and a variety of other health issues but hitherto has not been used in the form of nanoparticles or as a matrix or carrier for another drug. MMT not only cures minor problems, such as diarrhea and constipation through local application; it acts on all organs. Everything unhealthy, that emits negative radiations is irresistibly attracted to clay and becomes subject to immediate elimination (Lee and Chen, 2004). This invention recognizes such detoxification and body purification functions of MMT and uses it as a component of the matrix material for nanoparticle formulation of anticancer drugs for oral chemotherapy.

Another suitable candidate as the at least first drug is charcoal, or more specifically, activated charcoal.

Having now generally described the invention, the same will be more readily understood through reference to the following example which is provided by way of illustration, and are not intended to be limiting of the present invention.

### EXAMPLES

Preparation of Paclitaxel-Loaded MMT/PLGA Nanoparticles

Materials

Poly(D,L-lactide-co-glycolide) (PLGA, 50:50) and polyvinyl alcohol (PVA, Av. Mw 30,000-70,000) were purchased from Sigma. Paclitaxel was supplied by Yunan Hande Company, China. Montmorillonite (MMT, Closite Na⁺) was obtained from Southern Clay Products Incorporation. Dichloromethane (DCM), HPLC grade acetonitrile and fluorescence marker Coumarin-6 were purchased from Aldrich. All materials were used without further purification.

Methods

Paclitaxel-loaded MMT/PLGA nanoparticles were prepared by a modified solvent extraction/evaporation method. In a typical example, 5 mg paclitaxel and 110 mg PLGA were dissolved in 8 ml DCM. The resulted organic solution was first emulsified in 120 ml aqueous solution containing 2% w/v PVA and various amounts of MMT (0, 50%, 100%) and then sonicated for 120 s with the output power of 30 W of the sonicator. The organic solvent in the nanoemulsion was allowed to evaporate overnight at room temperature to harden the particles. The nanoparticle suspension was centrifuged, washed three times with deionized water and freeze-dried. Nanoparticles incorporated with 0, 50% and 100% MMT were labeled as NP, 50 MMT-NP and 100 MMT-NP. For fluorescence marking, Coumarin-6 was loaded in the MMT/PLGA nanoparticles. The preparation procedure was the same as above mentioned except that instead of the drug, 0.5% (w/v) Coumarin-6 was encapsulated in the nanoparticles.

As prepared by this method, the PLGA and MMT form a co-polymer, this co-polymer in turn forms the matrix of the nanoparticle. The paclitaxel is entrapped or encapsulated within this matrix. As the emulsifiers (PVA and unbound MMT) are removed, some MMT residue can remain on the surface of the nanoparticles.

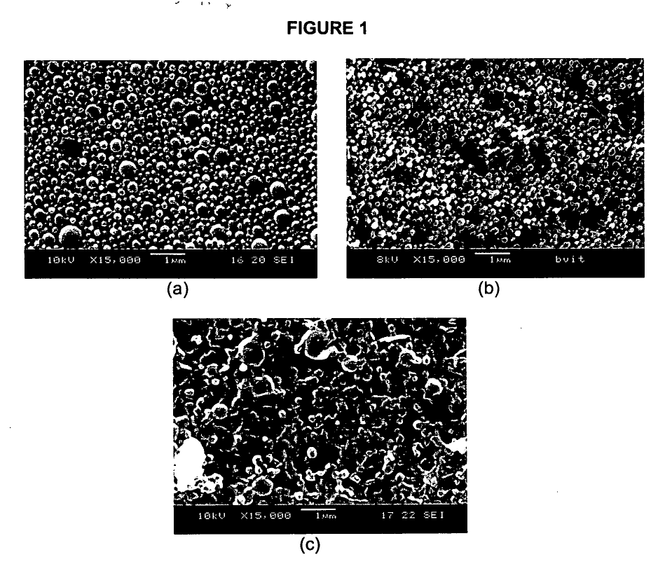

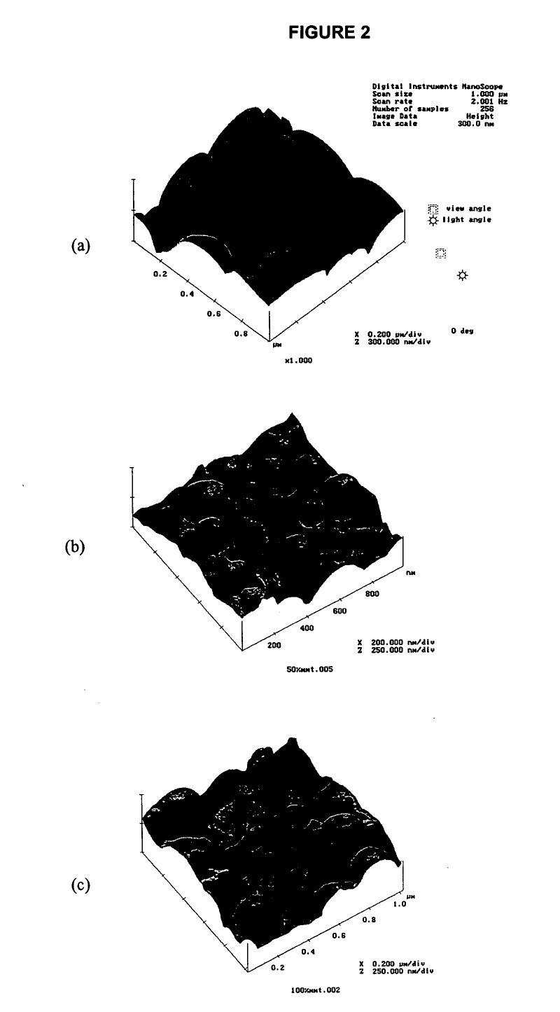

Thus prepared, the MMT may be present on the surface of the nanoparticles to varying degrees as may be seen in FIGS. 1 and 2. Where the MMT covers 50% or more of the surface area of a nanoparticle, the MMT is said to substantially cover or form the surface of a nanoparticle. Similary, where paclitaxel covers 50% or more of the surface area of a nanoparticle, the paclitaxel is said to substantially cover or form the surface of a nanoparticle.

The availability of the MMT on the surface of the nanoparticle confers desirable qualities on the paclitaxel loaded MMT/PLGA nanoparticles. The mucoadhesive property of the MMT allows the nanoparticles to be taken up into the cells and the MMT can control and/or reduce one or more side effects of the paclitaxel.

Characterization of Paclitaxel-Loaded MMT/PLGA Nanoparticles

(a) Measurement of Particle Size and Zeta Potential

The particle size and size distribution were measured by 90 Plus Particles Sizing (Brookhaven Instruments Corporation) based on the laser light scattering technique. Before measurement, the freshly prepared particle suspension was diluted to approximate 0.25 mg/ml. The size and size distribution of three samples of paclitaxel-loaded MMT/PLGA nanoparticles are shown in Table 1. All three formulations exhibited a size of around 310 nm with polydispersity less than 0.150. No significant difference in particle size and size distribution was observed among the three formulations with and without MMT. Among other process factors in the solvent/emulsion evaporation method, the surfactant used (PVA or MMT) plays an important role in reducing the particles size. At the interface between the oil phase and water phase, PVA or MMT stabilizes the formed nanoemulsion and prevents their aggregation. MMT is an excellent emulsion stabilizer, whose ability to stabilize the microemulsion is obtained by enveloping the particles or encapsulating the oil phase in the three-dimensional network of particles.

TABLE 1
Size, size distribution and zeta potential and drug encapsulation
efficiency of paclitaxel-loaded MMT/PLGA nanoparticles

| | Size | Polydispersity | Zeta potential | EE |
|---|---|---|---|---|
| Sample | (nm) | (%) | (mV) | (%) |
| NP | 304.3 | 0.131 | −18.84 | 50.5 |
| 50 MMT-NP | 310.2 | 0.141 | −24.89 | 46.5 |
| 100 MMT-NP | 310.9 | 0.144 | −27.27 | 52.0 |

Zeta potential or the surface charge of the formulated nanoparticles was determined by Zeta Potential Analyzer (Brookhaven Instruments Corporation). Zeta potential of the PLGA nanoparticles without MMT was measured to be −18.84 mV, which could be attributed to the carboxyl group in the PLGA polymer. The drug-loaded MMT/PLGA nanoparticles with 50% and 100% MMT showed more negative surface charge, i.e −24.89 and −27.27 mV, respectively. This could be ascribed to the presence of MMT, since MMT often carries negative charge due to the substitution of Al3+ for Si4+.

(b) Morphology

Morphology of the paclitaxel-loaded MMT/PLGA nanoparticles was chracterized by scanning electron microscope (SEM) (Jeol, JSM-5600 LV). Before the observation, the freeze-dried samples were coated by platinum for 40 s. Atomic Force Microscopy (AFM) (Mutimode Scanning Probe Microscope, Digital Instruments, Nanoscope IIIa) was also employed to observe the samples by the tapping mode. Before AFM observation, the particles were deposited and fixed on a double-sided sticky tape, which was then adhered to a sample stand. The paclitaxel-loaded MMT/PLGA nanoparticles were found by SEM to be spherical in shape and their size was around 300 nm (FIG. 1). The effects of presence of MMT on nanoparticle surface morphology can be observed from this figure. AFM images confirm the SEM findings in higher resolution (FIG. 2). It can be seen that the surface of the paclitaxel-loaded nanoparticles with MMT were obviously not as smooth as those without MMT, which indicates the presence of MMT on the nanoparticle surface.

(c) Drug Encapsulation Efficiency (EE)

The drug content in the MMT/PLGA nanoparticles was determined by HPLC. Typically, 3 mg of the freeze-dried drug-loaded MMT/PLGA nanoparticles were dissolved in 1 ml DCM. Upon the solvent evaporation, 5 ml of the 50% water/50% acetonitrile mixture were introduced to extract the paclitaxel inside the nanopartiles. The resulted solution was then analyzed by HPLC (Agilent LC 1100). The flow rate was set 1 ml/min and the detected wave length was 227 nm. The drug concentration can be calculated from the calibration curve. The EE is expressed as the percentage of the drug amount encapsulated in the nanoparticles to the added amount in the process. The drug encapsulation efficiency of samples NP, 50 MMT-NP and 100 MMT-NP were found to be 50.5%, 46.5% and 52.0%, respectively. The presence of MMT seems to have little influence on the EE though MMT helps stabilize the nanoemulsions.

Differential Scanning Calorimetry

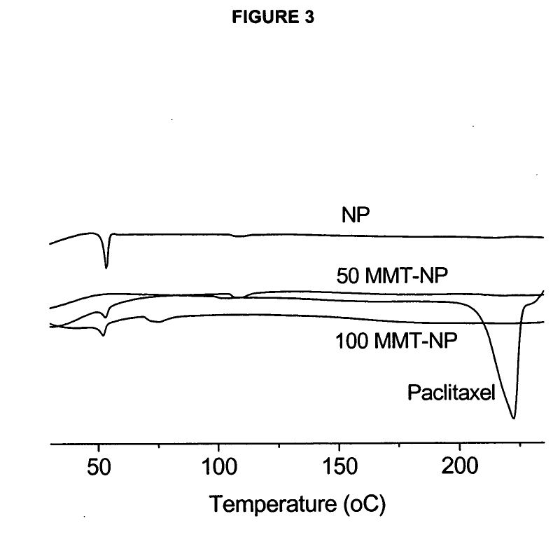

The physical status of the pure paclitaxel as well as the drug encapsulated in the MMT/PLGA nanoparticles was investigated by differential scanning calorimetry (DSC 822e, Mettler Toledo, Switzerland). The samples were purged with dry nitrogen with a flow rate of 20 ml/min. The temperature-rising speed was 10° C./min (FIG. 3). The melting endothermic peak of the pure paclitaxel appeared at 223° C. However, no melting peak was detected for the drug encapsulated in the MMT/PLGA nanoparticles. It can thus be concluded that paclitaxel in the MMT/PLGA nanoparticles was in an amorphous or disordered-crystalline phase or in the solid solution state. The curves of sample 100 MMT-NP also showed a weak endothermic peak at about 75 oC, which could be ascribed to the evaporation of free water in MMT.

(e) In Vitro Drug Release

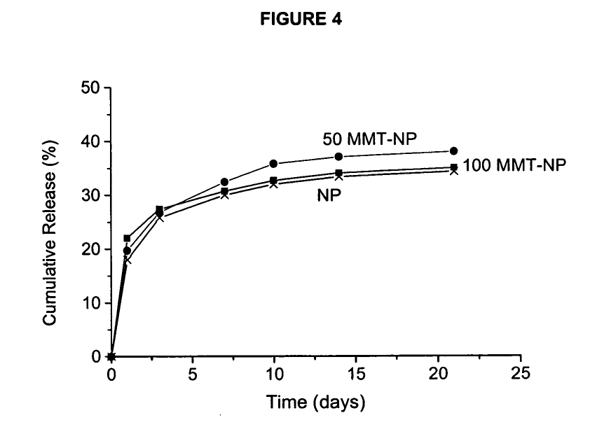

Five mg of the paclitaxel-loaded MMT/PLGA nanoparticles were suspended in 10 ml phosphate buffer solution (PBS, pH 7.4) in a capped centrifuge tube. The tube was placed in the water bath at 37° C. and shaken at the frequency of 120 Hz. At designated time intervals, the tube was taken out and centrifuged at 11,500 rpm for 15 minutes. The pellets were resuspended in 10 ml fresh PBS and put back to the shaker for continuous drug release study. The supernatant was collected and the drug inside was extracted with 1 ml DCM. After removal of DCM, the deposited paclitaxel was dissolved in 5 ml of the 50% water/50% acetonitrile mixture, followed by analysis steps as described for the EE measurement. The in vitro accumulated drug release profile of the paclitaxel-loaded MMT/PLGA nanoparticles is shown in FIG. 4. The initial burst of 22%, 20% and 18% in the first day can be observed for sample NP, 50 MMT-NP and 100 MMT-NP, respectively. After that, the release of paclitaxel was at a slower constant rate. In three weeks, about 36% drug was released for all the formulations. It appears that the drug release behaviour was not markedly affected by MMT. It should be pointed out that the drug release rate can be made fully under control by adjusting the composition parameters such as the type of polymer, the molecular weight of the polymer and the ratio of the copolymers and process parameters such as the emulsifier used, the mechanical strength of mixing, the oil-to-water phase ratio, the temperature and pH.

Cellular Uptake and Cell Viability of Paclitaxel-Loaded MMT/PLGA Nanoparticles

Caco-2 and HT-29 cells (American Type Culture Collection) were grown in 25 cm2 tissue culture flasks at 37° C. in 5% CO2 atmosphere. The medium, Dulbecco's Modified Eagle Medium (DMEM, Sigma D1152) supplemented with 10% fetal bovine serum, 100 U/ml penicillin and 100 ug/ml streptomycin (Sigma) was renewed every two days. After 90% confluence, the cells were collected by 0.25% trypsin-EDTA solution (Sigma) and cultured in 96-well black plate (Costar®, Corning Incorporated) at a density of 3×104 cells/well for investigation.

When the cells in 96-well plate reached about 90% confluence, the medium was removed. The cells were equilibrated with 100 μl Hanks Balanced Salt Solution (HBSS, GIBCO™, Invitrogen Corporation) for 1 hr. Upon removal of HBSS, 100 μl 0.25 mg/ml of the suspension of Coumarin 6-loaded nanoparticle of various amount of MMT were introduced to each well for fluorescence measurement to investigate cellular uptake of nanoparticles. The wells with no nanoparticles but with cells were used for negative control. For each sample, two columns of total 16 wells were used. The first column was used as positive control and the second one as samples. After 2 hrs incubation, the nanoparticle suspension was removed from the sample wells and washed three times with 100 ul PBS. 100 ul HBSS was introduced once again to the sample wells. Finally, 50 ul 0.5% Trixon-100/0.2 M NaOH were added to each well. The fluorescence intensity was quantitatively measured by a microplate reader. The excitation and emission wavelength were 430 and 485 nm, respectively. The cellular uptake of the MMT/PLGA nanoparticles was expressed as (Iₛₐₘₚₗₑ−I_{negative}/Iₚₒₛᵢₜᵢᵥₑ−I_{negative})×100%, where I is the fluorescence intensity. The Caco-2 cellular association of NP, 50 MMT-NP and 100 MMT-NP were 50%, 67% and 59%. The nanoparticle association was increased by 34% and 18%, respectively, due to the incorporation of 50% and 100% MMT in the formulation.

Similarly, in HT-29 cell line experiment, the presence of 50% and 100% MMT in the system resulted in an increase of the particle association from 56% to 80% and 67%, respectively (Table 2). The cellular association assay shows that incorporation of MMT in the nanoparticle formulation can significantly enhance the interactions between the nanoparticles and the cells. The enhanced interaction may be resulted in part from the increased viscosity of the particle suspension, which may help the nanoparticles to associate with the cells/mucus. But more possibly, MMT may develop London-van der Vaals forces and hydrogen bonding with the cells (Lavie and Stotzky, 1996). Suspended together with MMT, the nanoparticles may be surrounded and adsorbed by MMT, which acts as adhesives between the particles and the cells. Hydrogen bonding may be formed between the hydroxyl groups of glycolproteins and the water of hydration of cations on the MMT.

The involved London-van der Vaals forces and hydrogen bonding developed may be mainly responsible for the increased particles-cells interactions. It was also found that 100% MMT resulted in less particle association with Caco-2 and HT-29 cells than 50% MMT did. This could be due to the more viscous suspension of higher amount of MMT formulation, in which the transport of the nanoparticles might become difficult.

TABLE 2
Cellular association of paclitaxel-loaded MMT/PLGA nanoparticles
with Caco-2 and HT-29 cells

| | Cellular Association (%) | |
|---|---|---|
| Sample | Caco-2 | HT-29 |
| NP | 50% | 56% |
| 50 MMT-NP | 67% | 80% |
| 100 MMT-NP | 59% | 67% |

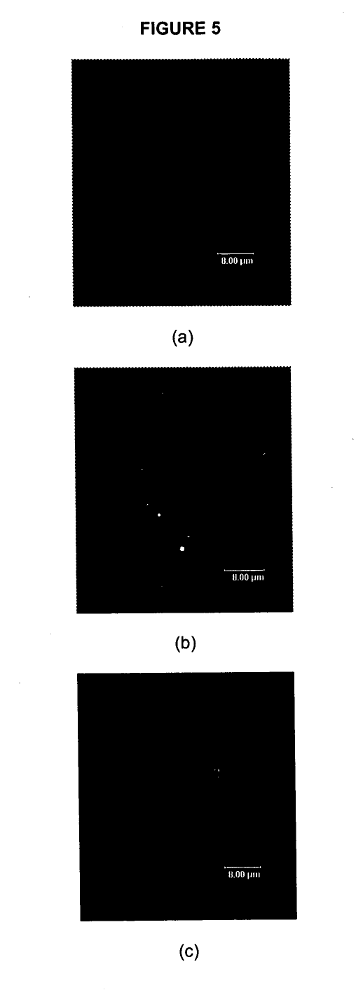

For confocal imaging, Caco-2 cells were grown on the chambers (Lab-Tek® Chambered Coverglass System) and maintained at 37° C. and 5% CO2. After 90% confluence, the medium was removed. Caco-2 cell monolayers were incubated with 500 μl 0.25 mg/ml nanoparticle suspension for 2 hrs. Upon removal of the free nanoparticles by washing the samples thrice with PBS, the cells were fixed by a certain amount of ethanol and incubated for 20 minutes. Subsequently, propidium iodide was added to stain the nucleus after the removal of ethanol. Finally, the cells were mounted by the mounting medium (DAKO® Fluorescent Mounting Medium) and observed under confocal laser scanning microscopy (Leica TCS SP2) using FITC filter. The cells without nanoparticles incubation were used as control. Confocal images of the Caco-2 cells after incubation with 0.25 mg/ml fluorescent nanoparticle suspension for 2 hrs are shown in FIG. 5. Apparent fluorescence in the cell cytoplasm can be observed in comparison with the controls, indicating that the fluorescent nanoparticles have been internalized by the cells.

Upon oral administration, the particles may undergo (1) absorption by the intestinal cells, (2) adhesion to the mucus/epithelial cells and releasing the encapsulated drug at the anchoring sites, and (3) elimination. High drug bioavailability requires reducing the direct elimination of the drug loaded-nanoparticles in the GI tract. Hence, the strong interactions between the nanoparticles and the GI tract mucus/epithelial cells are essential to prolong the residence time and thus enhance the absorption of the drug-loaded nanoparticles. The intestine epithelium is composed of absorptive enterocytes and other cells such as goblet cells and M cells. Caco-2 and HT-29 in vitro cell culture systems are both derived from human colon carcinoma. Enterocyte-like Caco-2 cell lines are widely used to simulate the GI barrier to investigate the uptake and transport of the drugs and drug loaded nanoparticles since this kind of cells shows the similar properties with intestinal epithelial cells, while HT-29 cells upon culture represent the second major type of intestinal cells (Artursson et al, 2001). Our results thus show the feasibility for MMT/PLGA nanoparticles to deliver drugs across the GI barrier for oral chemotherapy.

Variations Under the Present Invention

A person skilled in the art will appreciate that many other variations of the present invention may be possible. Such variations may include, but are not be confined to:

Nanoparticle formulation of other therapeutic effects,

Oral delivery of other therapeutic agents,

Nasal delivery of therapeutic agents,

Ocular delivery of therapeutic agents,

Local delivery of therapeutic agents,

Gene delivery, and

Delivery of contrast materials for MRI.

It will be appreciated that various modifications and improvements can be made by a person skilled in the art without departing from the scope of the present invention.

### REFERENCES

Artursson P, Palm K, Luthman K. Caco-2 monolayers in experimental and theoretical predictions of drug transport, Adv. Drug Deliv. Rev. 46, 2743 (2001).
Bummer P M. Physical chemical considerations of lipid-based oral drug delivery—Solid lipid nanoparticles. Crit Rev Therap Drug Carrier Syst 21 (1): 1-19 (2004).
Feng S S. Nanoparticles of Biodegradable Polymers for New Concept Chemotherapy. Expert Review of Medical Devices. 1(1):89-99 (2004)
Lavie S, Stotzky G. Adhesion of the clay minerals montmorillonite, kaolinite, and attapulgite reduces respiration of Histoplasma capsulate. Appl. Environ. Microbio. 51 (1), 65-73 (1986).
Lee W F, Chen Y C. Effect of bentonite on the physical properties and drug-release behavior of poly(AA-co-PEGMEA)/bentonite nanocomposite hydrogels for mucoadhesive. J Applied Polymer Sci 91 (5): 2934-2941 (2004).
Malingre M M, Beijnen J H, Schellens J H M. Oral delivery of taxanes. Invest New Drug, 19(2), 155-162 (2001A).

What we claim is:

1. A drug delivery system comprising at least one type of nanoparticle, at least one first drug, and at least one second drug, wherein the nanoparticle and the first drug are contacted and the second drug is applied on the contacted nanoparticle and at least one first drug, and wherein the at least one first drug possesses at least one of the following properties:
(a) emulsifier;
(b) mucoadhesion; or
(c) controls and/or reduces at least one side effect of the at least one second drug.

2. The drug delivery system according to claim 1, wherein the contacted nanoparticle and the at least one first drug form a matrix or part thereof.

3. The drug delivery system according to claim 1, wherein the at least one first drug substantially covers the surface of the nanoparticle.

4. The drug delivery system according to claim 1, wherein the at least one second drug substantially covers the surface of the nanoparticle.

5. The drug delivery system according to claim 1, wherein the at least one first drug is MMT.

6. The drug delivery system according to claim 1, wherein the at least one second drug is a paclitaxel.

7. The drug delivery system according to claim 1, wherein the at least one type of nanoparticle is biodegradable and/or bioresorbable.

8. The drug delivery system according to claim 7, wherein the at least one type of nanoparticle is PLGA.

9. The drug delivery system according to claim 1, wherein the drug delivery system is for delivering at least one drug across a mucosal membrane.

10. The drug delivery system according to claim 9, wherein the mucosal membrane is the lining of the gut.

11. The drug delivery system according to claim 9, wherein the mucosal membrane is the lining of the respiratory tract.

12. A method of delivering at least one drug across a mucosal membrane in a subject, the method comprising:
providing a drug delivery system comprising at least one type of nanoparticle, at least one first drug, and at least one second drug, wherein the nanoparticle and the first drug are contacted and the second drug is applied on the contacted nanoparticle and at least one first drug, and wherein the at least one first drug possesses at least one of the following properties:
(a) emulsifier;
(b) mucoadhesion or
(c) controls and/or reduces at least one side effect of the at least one second drug;
and
applying the delivery system to the musosal membrane.

13. The method according to claim 12, wherein the contacted nanoparticle and the at least one first drug form a matrix or part thereof.

14. The method according to claim 12, wherein the at least one first drug substantially covers the surface of the nanoparticle.

15. The method according to claim 12, wherein the at least one second drug substantially covers the surface of the nanoparticle. The drug delivery system according to claim 12, wherein the at least one first drug is MMT.

16. The method according to claim 12, wherein the at least one second drug is a paclitaxel.

17. The method according to claim 12, wherein the at least one type of nanoparticle is biodegradable and/or bioresorbable.

18. The method according to claim 17, wherein the at least one type of nanoparticle is PLGA.

19. The method according to claim 12, wherein the mucosal membrane is the lining of the gut.

20. The method according to claim 12, wherein the mucosal membrane is the lining of the respiratory tract.

21. A drug delivery system comprising at least one type of nanoparticle, at least one first drug, and at least one second drug, and wherein the at least one second drug is substantially applied on the surface of the nanoparticle and the at least one first drug is substantially applied on the second drug, and wherein the at least one first drug possesses at least one of the following properties:
(a) emulsifier;
(d) mucoadhesion, or
(e) controls and/or reduces at least one side effect of the at least one second drug.

22. The drug delivery system according to claim 21, wherein the at least one first drug is MMT.

23. The drug delivery system according to claim 21, wherein the at least one second drug is a paclitaxel.

24. The drug delivery system according to claim 21, wherein the at least one type of nanoparticle is biodegradable and/or bioresorbable.

25. The drug delivery system according to claim 24, wherein the at least one type of nanoparticle is PLGA.

26. The drug delivery system according to claim 21, wherein the drug delivery system is for delivering at least one drug across a mucosal membrane.

27. A method of delivering at least one drug across a mucosal membrane in a subject, the method comprising:
providing a drug delivery system comprising at least one type of nanoparticle, at least one first drug, and at least one second drug, and wherein the at least one second drug is substantially applied on the surface of the nanoparticle and the at least one first drug is substantially applied on the second drug, and wherein the at least one first drug possesses at least one of the following properties:
(a) emulsifier;
(b) mucoadhesion or
(c) controls and/or reduces at least one side effect of the at least one second drug;
and
applying the delivery system to the musosal membrane.
